(19) United States
(12) Patent Application Publication
(10) Pub. No.: US 2022/0378704 A1
(43) Pub. Date: Dec. 1, 2022

(54) PROCESS FOR THE PREPARATION OF A NANOPARTICULATE ACTIVE INGREDIENT

(71) Applicant: Bayer Aktiengesellschaft, Leverkusen (DE)

(72) Inventors: Qingbai CAI, Shanghai, Free Trade Zone (CN); Leslaw MLECZKO, Dormagen (DE); Werner HOHEISEL, Koeln (DE); Shizhe TIAN, Shanghai, Free Trade Zone (CN); Min FU, Shanghai, Free Trade Zone (CN)

(21) Appl. No.: 17/642,043

(22) PCT Filed: Oct. 5, 2020
(86) PCT No.: PCT/EP2020/077776

§ 371 Date: Mar. 10, 2022

(30) Foreign Application Priority Data
Oct. 10, 2019 (CN) PCT/CN2019/110379

(51) Int. Cl. A61K 9/16

(52) U.S. Cl. CPC A61K 9/1688; A61K 9/1635

(57) ABSTRACT

A process for the preparation of a nanoparticulate active ingredient comprises the steps of: a) providing a solvent, a pharmaceutical active ingredient dissolved in the solvent, a liquid antisolvent and a stabilizer which is dissolved in the solvent or in the antisolvent and wherein the antisolvent is miscible in the solvent; b) mixing the solvent, the active ingredient, the antisolvent and the stabilizer in a micromixer, thereby obtaining a suspension comprising a precipitate of the active ingredient, the solvent and the antisolvent. The active ingredient precipitate is present in the form of nanoparticles having an average particle size of ≥10 nm to ≤999 nm and a particle size distribution, determined by dynamic light scattering (DLS) according to ISO 22412:2017, having a polydispersity index of ≤0.2.

The present invention relates to a process for the preparation of a nanoparticulate active ingredient comprising the steps of: a) providing a solvent, a pharmaceutical active ingredient dissolved in the solvent, a liquid antisolvent and a stabilizer which is dissolved in the solvent or in the antisolvent and wherein the antisolvent is miscible in the solvent; b) mixing the solvent, the active ingredient, the antisolvent and the stabilizer in a micromixer, thereby obtaining a suspension comprising a precipitate of the active ingredient, the solvent and the antisolvent.

A high bioavailability and short dissolution times are desirable attributes of a pharmaceutical end product. It has been estimated that 40% or even more of drugs in the pharmaceutical industry fall into the biopharmaceutical class II (low solubility-high permeability) and class IV (low solubility-low permeability) categories. Among various strategies to address the solubility issue, reducing the drug particle sizes has emerged as an effective and versatile option.

Many top-down and bottom-up methods such as ultrafine mechanical milling, spray drying and high-pressure homogenization were developed to generate micro- or nanodrug particles. Nevertheless, they still have some limitations, such as high energy input, low yield, pharmaceutical contaminants and difficult to control particle size and surface properties which restrict their applications and further commercialization.

Precipitation processes as described above are typically used to produce micro- and nanosized particles.

A reactor with excellent mixing properties is expected to be beneficial to forming uniform drug nanoparticles. A microchannel reactor (MCR) provides a reaction volume and a microchannel that is more homogenous with respect to concentration, temperature, mass transfer and heat transfer, leading to a better control of the reaction or the precipitation step that governs the particle size and its distribution, i.e. nucleation and growth.

Therefore, an MCR appears to be a promising platform for drug micronization. Precipitations combined with MCR technology to produce micro- and nanosized particles have been described in various publications with average particle diameters reported in the range of 100 nm to 80 μm.

Hong Zhao et al., Ind. Eng. Chem. Res. 2007, 46, 8229-8235, propose liquid antisolvent precipitation (LASP) process in a microchannel reactor (MCR) to directly synthesize danazol nanoparticles without additives. The mean particle size reportedly decreased from 55 μm to 364 nm, and the specific surface area reportedly increased from 0.66 to 14.37 m²/g after the LASP process in the MCR. The mean particle size is reported to be facilely controlled by regulating the parameters of the MCR setup. The chemical composition and physical characteristics of the as-prepared danazol nanoparticles reportedly were demonstrated to be unchanged after processing according to FT-IR and XRD analyses. Correspondingly, the dissolution rate of the nanoparticles within 5 min reportedly was enhanced from 35% to 100% compared to the raw danazol particles.

C. Beck et al., Chemical Engineering Science 2010, 65, 21, 5669-5675, report that particle formation by the liquid antisolvent (LAS) process involves two steps: mixing of solution-antisolvent streams to generate supersaturation and precipitation (which includes nucleation and growth by coagulation and condensation) of particles. Uniform mixing conditions reportedly ensure rapid and uniform supersaturation, making it a precipitation controlled process where the particle size is not further affected by mixing conditions and results in precipitation of ultra-fine particles with narrow particle size distribution (PSD). In this work, the authors reported that the use of an ultrasonically driven T-shaped mixing device significantly improves mixing of solution and antisolvent streams for precipitation of ultra-fine particles in a continuous operation mode. LAS precipitation of ultra-fine particles of multiple active pharmaceutical ingredients (APIs) such as itraconazole (ITZ), ascorbyl palmitate (ASC), fenofibrate (FNB), griseofulvin (GF), and sulfamethoxazole (SFMZ) in the size range 0.1-30 μm has reportedly been carried out from their organic solutions in acetone, dimethyl sulfoxide (DMSO), tetrahydrofuran (THF), and ethanol (EtOH). Classical theory of homogeneous nucleation has been used to analyze the result, which reportedly suggests that higher nucleation rate results in finer particle size. Reportedly, experimental determination of degree of supersaturation indicates that higher supersaturation does not necessarily result in higher nucleation rate and nucleation rates can be correlated to solvent polarity.

Qianxia Zhang et al., Journal of Chemical Industry and Engineering (China) 2011, 7, report that as model drug, beclomethasone dipropionate (BDP) nanoparticles were prepared by using the antisolvent precipitation method in a T-junction microchannel. The influence of surfactant on particle morphology, as well as the influences of surfactant concentration, BDP solution flow rate, antisolvent flow rate, BDP solution concentration and precipitation temperature on particle size were explored. The results were reported to indicate that the morphology of BDP was spherical with the addition of surfactant (HPMC). Besides, the particle size reportedly decreased with decreasing BDP solution flow rate, increasing antisolvent flow rate and decreasing precipitation temperature. However, with the increase of BDP concentration, particle size reportedly reached a minimum. BDP nanoparticles with an average size of 200-260 nm and narrow size distribution reportedly could be prepared under the following conditions: BDP solution flow rate of 4 ml/min, antisolvent flow rate of 80 ml/min, solution concentration of 0.03 g/ml. Furthermore, crude BDP and nanosized BDP were characterized by scanning electronic microscopy (SEM), X-ray diffraction (XRD), Fourier transform infrared spectrophotometry (FTIR), and surface area analyzer. The results reportedly showed that the precipitated BDP without HPMC was crystalline while the precipitated BDP with HPMC was completely amorphous. In addition, the as-prepared BDP reportedly had the same molecular structure as raw drug, and had a specific surface area 2 times as high as that of the raw drug. A T-junction microchannel reportedly would become a high-efficiency, low-cost technology platform for preparing nanodrugs.

US 2005/0206022 A1 discloses a process for the preparation of small particles through precipitation. The patent application relates to such a process which employs a fluid solution comprising a solvent and solute to be precipitated and a non-gaseous antisolvent, said solvent being soluble in or miscible with the antisolvent and said solute being substantially insoluble in the antisolvent, wherein the process comprises the successive steps of: feeding a stream of a fluid solution and a stream of the antisolvent into a mixing zone where both streams are thoroughly mixed to achieve a condition of super saturation while ensuring that hardly any nucleation occurs during the mixing; feeding the resulting mixture of the fluid solution and the antisolvent into a nucleation zone allowing nucleation to commence; allowing the nuclei formed in the nucleation zone to grow to particles with a volume weighted average diameter of no more than 50 μm, preferably of no more than 7 μm; and collecting the particles and separating them from the antisolvent.

EP 1 423 096 B1 relates to a process for preparing crystalline particles of a drug substance, said process comprising recirculating an anti-solvent through a mixing zone, dissolving the drug substance in a solvent to form a solution, adding the solution to the mixing zone to form a particle slurry in the anti-solvent, and recirculating at least a portion of the particle slurry back through the mixing zone. Particles produced from the process are also disclosed. The disclosed invention reportedly has the ability to be operated in a continuous fashion, resulting in a more efficient process and a more uniform product. The present invention reportedly has the additional advantage of having the ability to operate at a relatively low solvent ratio, thereby increasing the drug to excipient ratio.

US 2013/0012551 A1 describes a method for producing microparticles or nanoparticles of water-soluble and water-insoluble substances by controlled precipitation, co-precipitation and self-organization processes in microjet reactors, a solvent, which contains at least one target molecule, and a nonsolvent being mixed as jets that collide with each other in a microjet reactor at defined pressures and flow rates and thereby effect very rapid precipitation, co-precipitation or a chemical reaction, during the course of which microparticles or nanoparticles are formed. In order to create such a method, with which the particle size of the resulting microparticles or nanoparticles can be specifically controlled, it is proposed that particle size be controlled by the temperature at which the solvent and nonsolvent collide, the flow rate of the solvent and the nonsolvent and/or the amount of gas, smaller particle sizes being obtained at lower temperatures, at high solvent and nonsolvent flow rates and/or in the complete absence of gas.

The above-mentioned methods of preparing micro- or nanoparticles suffer from the drawbacks that 1) mixing, nucleation and particle growth occur simultaneously, nuclei may form in the mixing zone, which leads to a large particle size distribution (PSD) and particle size; 2) high residence times are sometimes needed to prepare nanoparticles, which in turn will increase the particle size; 3) a high fluid velocity is also needed to achieve jetting conditions, necessitating high energy input and 4) other factors employed in the processes, such as ultrasound technology or supercritical fluid technology are cost-intensive.

The present invention has the object of at least partially overcoming the drawbacks of the prior art. In particular the object is to provide a process for preparing re-dispersible drug nanoparticles.

This object has been achieved by a process according to claim 1. Advantageous embodiments are the subject of the dependent claims. They may be combined freely unless the context clearly indicates otherwise.

Accordingly, a process for the preparation of a nanoparticulate active ingredient comprises the steps of:

a) providing a solvent, a pharmaceutical active ingredient dissolved in the solvent, a liquid antisolvent and

a stabilizer which is dissolved in the solvent or in the antisolvent and wherein the antisolvent is miscible in the solvent,

b) mixing the solvent, the active ingredient, the antisolvent and the stabilizer in a micromixer, thereby obtaining a suspension comprising a precipitate of the active ingredient, the solvent and the antisolvent,

wherein the active ingredient precipitate is present in the form of nanoparticles having an average particle size of ≥10 nm to ≤999 nm and a particle size distribution, determined by dynamic light scattering (DLS) according to ISO 22412:2017, having a polydispersity index of ≤0.2 (preferably >0 to ≤0.15, more preferred >0 to ≤0.1).

If desired, the process may also include the following step after step b):

c) removing the solvent and the antisolvent from the suspension.

The process according to the invention may be, without wishing to be limited, described as a process for preparing nanoparticles of a drug using an antisolvent precipitation method carried out in a microchannel device such as a valve-assisted micromixer, cascade micromixer, LH type micromixer, etc. These devices provide a better micro-mixing effect, thereby further narrowing the particle size distribution and particle size.

A feature of micromixers is the small dimensions of the fluid channels, which typically lie in the range of 10 to 5000 μm. For this reason, for example with multi-lamination mixers, it is possible to generate fine fluid lamellae between which rapid substance exchange can take place by diffusion owing to their small thickness. Preferably the micromixers comprise mixing plates with nominal slit diameters between 100 and 400 μm.

A one-step continuous process based on the homogeneous nucleation mechanism is more efficient, results in a more uniform product and smaller mean particle sizes (for example, ca. 65 nm). The process has the additional advantage of enabling smaller residence times in the microchannel device (e.g., 0.08 to 0.09 seconds) without other high energy input at atmospheric pressure. The process according to the invention may be operated at a wide range of solvent/antisolvent ratios.

The pharmaceutical active ingredient, preferably one for oral administration, can be selected from a variety of known classes of drugs, including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiacinotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, and combinations thereof.

The pharmaceutical active ingredient may in particular be a non-steroidal anti-inflammatory drug (NSAID), for example acetylsalycic acid derivatives, arylpropionic acid derivatives, arylacetic acid derivatives, indoleacetic acid derivatives, anthranilic acid derivatives, oxicams and selective COX-2 inhibitors.

Specific examples include alminoprofen, benoxaprofen, bucloxic acid, carprofen, fenbufen, fenoprofen, fluorophen, flurbiprofen, ibuprofen, indoprofen, ketoprofen, miroprofen, naproxen, oxaprozin, pirprofen, pranoprofen, suprofen, tiaprofenic acid, tioxaprofen, indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, zomepirac, flufenamic acid, meclofenamic acid, mefenamic acid, niflumic acid, tolfenamic acid, diflunisal, flufenisal, isoxicam, piroxicam, sudoxicam, tenoxican, acetyl salicylic acid, sulfasalazine, apazone, bezpiperylon, feprazone, mofebutazone, oxyphenbutazone, phenylbutazone, vericiguat, riociguat or a mixture of at least two of the aforementioned substances. Particularly preferred are indomethacin, naproxen and vericiguat.

Generally, polymeric stabilizers are preferred. Examples of particle stabilizers include phospholipids, surfactants, polymeric surfactants, vesicles, polymers, including copolymers and homopolymers and biopolymers, and/or dispersion aids. Suitable surfactants include gelatin, casein, lecithin, (phosphatides), gum acacia, cholesterol, tragacanth, stearic acid, benzalkonium chloride, calcium stearate, glyceryl monostearate, cetostearl alcohol, cetomacrogol 1000, polyoxyethylene castor oil derivatives, polyoxyethylene sorbitan fatty acid esters, for example, the commercially available Tweens, polyethylene glycols, poly(ethylene oxide/propylene oxide) copolymers, for example, the commercially available Poloxamers or Pluronics, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose, noncrystalline cellulose, magnesium aluminum silicate, triethanolamine, polyvinylalcohol, sodium lauryl sulfate, polyvinylpyrrolidone (PVP), poly(acrylic) acid, and other anionic, cationic, zwitterionic and nonionic surfactants. Other suitable stabilizers are described in detail in the Handbook of Pharmaceutical Excipients, published jointly by the American Pharmaceutical Association and The Pharmaceutical Society of Great Britain, the Pharmaceutical Press, 1986. Such stabilizers are commercially available and/or can be prepared by techniques known in the art. The weight ratio of drug to stabilizer may vary but is typically in a range of 1:100 to 100:1.

Particularly preferred are polyvinylpyrrolidone (especially PVP K12 and PVP K30 grades) and N-vinylpyrrolidone-vinylacetate-copolymers.

Suitable organic solvents include but are not limited to methanol, ethanol, isopropanol, 1-butanol, t-butanol, trifluoroethanol, polyhydric alcohols such as propylene glycol, PEG 400, and 1,3-propanediol, amides such as n-methyl pyrrolidone, n,n-dimethylformamide, tetrahydrofuran, propionaldehyde, acetone, n-propylamine, isopropylamine, ethylene diamine, acetonitrile, methyl ethyl ketone, acetic acid, formic acid, dimethylsulfoxide, 1,3-dioxolane, hexafluoroisopropanol, and combinations thereof.

The concentration of the drug dissolved in the solvent is preferably as close as practical to the solubility limit of the solvent at room temperature. Such concentration will depend upon the selected drug and solvent but is typically in the range of from 0.1 to 20.0 weight percent.

The anti-solvent is a liquid which is at least partially miscible with the solvent and in which the pharmaceutical active ingredient is practically insoluble, for example having a solubility of less than 10 mg/ml, preferably less than 5 mg/ml and more preferred less than 1 mg/ml. Preferably the selected solvent exhibits ideal mixing behaviour with the anti-solvent so that the solution can be instantaneously distributed through the resulting particle suspension.

According to an embodiment of the process the nanoparticulate active ingredient has an average particle size, determined by dynamic light scattering (DLS) according to ISO 22412:2017, of ≥20 to ≤300 nm, preferably ≥10 to ≤200 nm.

According to another embodiment of the process the nanoparticulate active ingredient has a spherical or nearly spherical shape with an aspect ratio of ≤2:1, preferably ≤5:1.

According to another embodiment of the process the flow in the micromixer is turbulent and the Reynold number Re is ≥2300, preferably Re ≥10000 and particularly preferably Re ≥20000.

According to another embodiment of the process in the micromixer the segregation index Xs is ≤0.1, preferably ≤0.02, particularly preferred Xs is ≤0.01 and most particularly preferably Xs is ≤0.005.

According to another embodiment of the process the micromixer is a valve-assisted mixer or a cascade mixer. In a valve-assisted mixer a non-return valve can almost totally prevent any back-flow of the mixture. Preferred valve-assisted micromixers are mixers having a first channel for supplying a first sub-flow and having a second channel for supplying a second sub-flow, which open in flat, preferably narrow entry gaps into a mixing and reaction zone and leave the mixing and reaction zone via an outlet channel, wherein a reflux barrier is arranged between the mixing and reaction zone and at least one channel for supplying a sub-flow. One of the entry gaps preferably has a reflux barrier formed in the region where it opens into the reaction zone. Such micromixers are, inter alia, described in WO 2005/079964 A1.

The mixing principle of a cascade mixer is a split-and-recombine operation. Preferred is a static micromixer with supply chambers for at least two fluids to be mixed, from which microchannels lead to a mixing chamber, wherein said microchannels are arranged in at least two adjacent supply elements, wherein the supply elements are wedge-shaped plates, which can be assembled to form at least one ring sector that surrounds the mixing chamber in a curve, and the microchannels provided for each fluid form a symmetrical bifurcation cascade comprising at least two stages. Such cascade mixers are, inter alia, described in WO 2001/043857 A1.

According to another embodiment of the process the active ingredient has a solubility in the mixture of solvent, antisolvent and stabilizer of ≤1% by weight based on the total mass, preferably ≤0.1% by weight, particularly preferably ≤0.01% by weight.

According to another embodiment of the process the antisolvent is water.

According to another embodiment of the process the water has a temperature of >0-≤30° C., preferably >0-≤10° C., particularly preferred >0-≤5° C.

According to another embodiment of the process the solvent is an alkanol. Examples include methanol, ethanol, isopropanol, n-propanol and mixtures containing at least two of the aforementioned compounds.

According to another embodiment of the process the ratio of the solvent to the antisolvent is ≥1:100, preferably ≥1:40, more preferably ≥1:20, most preferably ≥1:10, most preferably ≥1:5 and particularly preferred ≥1:1.

According to another embodiment of the process the flow rate of the solvent with the dissolved active ingredient and the flow rate of the antisolvent are in a ratio of ≥1:100 to ≤1:1. Preferred rations are ≥1:60 to ≤1:1, more preferred ≥1:5 to ≤1:1.

According to another embodiment of the process the concentration ratio of stabilizer to active ingredient in the mixture is ≤10:1, preferably ≤5:1, particularly preferably ≤2:1 and very particularly preferably ≤1:2.

According to another embodiment of the process the stabilizer is polyvinylpyrrolidone.

According to another embodiment of the process the weight ratio of active ingredient to stabilizer is in a range of ≥1:1 to ≤5:1. Preferred rations are ≥2:1 to ≤5:1, more preferred ≥4:1 to ≤5:1.

Particularly preferred is the combination of naproxen, ethanol as a solvent and water as an anti-solvent.

The present invention will be further described with reference to the following examples and figures without wishing to be limited by them.

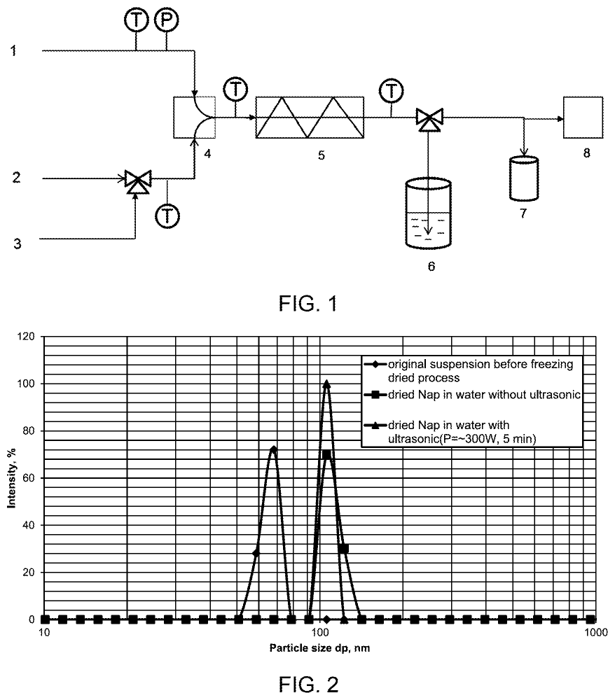

FIG. 1 shows a configuration for carrying out the method according to the invention. Depicted are a drug solvent infeed 1, an anti-solvent infeed 2, a cleaning solvent infeed 3, a mixer 4 for mixing the solvent and the antisolvent, a microchannel reactor 5 for nucleation and particle growth, a waste vessel 6, a sample vessel 7, a post-treatment device 8, temperature sensors T and pressure sensors P.

The solvent infeed 1 and the anti-solvent infeed 2 are preferably ideally mixed with each other in the mixer 4. Then the mixture flows into the microchannel reactor 5 where the nucleation and particle growth occurs. The particle suspension can then be fed into sample vessel 7 to measure the target particle size to check whether the particle size or other quality parameters are met. If not, the suspension will be discarded into the waste vessel 6. The obtained suspension can be treated according to the drug dosage requirement. For example, if the delivery system needs a powdered drug, the nanoparticle suspension will flow into the post-treatment device 8 such as a freeze-dryer, spray dryer, film evaporator, and the like in order to remove the solvents and to obtain the dry nanoparticle powder. If the dosage form is a nanoparticle suspension the original suspension would be diluted or concentrated to obtain the proper concentration of the drug suspension. Optionally, the cleaning solvent infeed 3 is designed to dissolve any drug particles which may block the mixer 4, the reactor 5 or any other auxiliary facilities.

### Example 1

A continuous liquid antisolvent precipitation process as outlined in FIG. 1 was used. The micromixer, a valve mixer, was a Modular MicroReaction System by Ehrfeld Mikrotechnik GmbH, Germany. Deionized water was used as an antisolvent in the process and ethanol as a solvent. Firstly, deionized water continuously flowed in the system at a flow rate of 80 ml/min at a constant temperature of 20° C. by means of an HPLC constant-flow pump. 50.0 grams of a solvent solution of 5.0 weight-% naproxen and 2.5 weight-% polyvinylpyrrolidone (PVP) K30 in ethanol were pumped at a volume flow rate of 1 ml/min at a temperature of 1° C. by an HPLC constant-flow pump. A nanoparticle suspension was formed by mixing the solvent and the antisolvent in the valve mixer used in the example. The particle size distribution of the particles in suspension was measured without filtration using a Malvern Nano series ZSP analyzer (dynamic light scattering) and is given in FIG. 2 (“Nap”: naproxen particles). The particle suspension was then introduced into a freezing dryer at a temperature of −50° C. to −60° C. The precipitation experiment was repeated several times. The particle sizes dp (arithmetic mean particle diameter) after freeze drying were 109.2 nm (without ultrasonic treatment) and 106.3 nm (with ultrasonic treatment), respectively. As the example shows, the particle size did not change after ultrasonic treatment of the sample. The PDI value after precipitation was around 0.18. The PDI value after freeze drying without ultrasonication was around 0.16 and with ultrasonication 0.15.

### Example 2

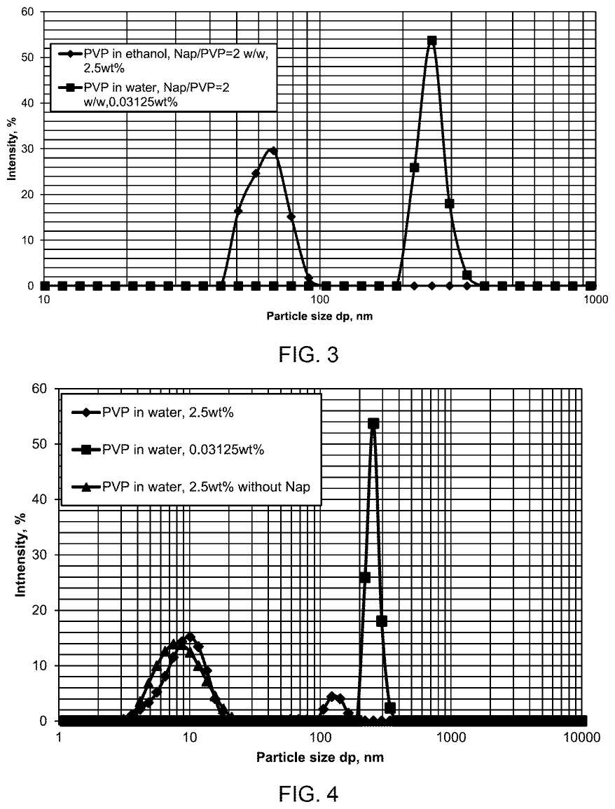

The stabilizer PVP K30 was added to the anti-solvent water (0.03125 weight-%) with the same weight ratio of drug to stabilizer (2:1) as in example 1. The other experimental conditions were also the same. The mean particle size in nanosuspension without filtration was about 225.0 nm (Malvern Nano series ZSP) and the particle size distribution is depicted in FIG. 3 (“Nap”: naproxen particles).

### Example 3

The concentration of the stabilizer PVP K30 added into the water was changed from 0.03125 weight-% to 2.5 weight-%. The other conditions were the same as in example 2. The mean particle size in nanosuspension without filtration was about 122.4 nm and the particle size distribution is depicted in FIG. 4 (“Nap”: naproxen particles).

1. A process for preparation of a nanoparticulate active ingredient comprising:
a) providing a solvent, a pharmaceutical active ingredient dissolved in the solvent, a liquid antisolvent and
a stabilizer which is dissolved in the solvent or in the antisolvent and wherein the antisolvent is miscible in the solvent,
b) mixing the solvent, the active ingredient, the antisolvent and the stabilizer in a micromixer, thereby obtaining a suspension comprising a precipitate of the active ingredient, the solvent and the antisolvent,
wherein
the active ingredient precipitate is present in the form of nanoparticles having an average particle size of ≥10 nm to ≤999 nm and a particle size distribution, determined by dynamic light scattering (DLS) according to ISO 22412:2017, having a polydispersity index of ≤0.2.

2. The process according to claim 1, wherein the nanoparticulate active ingredient has an average particle size, determined by dynamic light scattering (DLS) according to ISO 22412:2017, of ≥20 to ≤300 nm.

3. The process according to claim 1, wherein the nanoparticulate active ingredient has a spherical or nearly spherical shape with an aspect ratio of ≤2:1.

4. The process according to claim 1, wherein the flow in the micromixer is turbulent and the Reynold number Re is ≥2300.

5. The process according to claim 1, wherein in the micromixer the segregation index Xs is ≤0.1.

6. The process according to claim 1, wherein the micromixer is a valve-assisted mixer or a cascade mixer.

7. The process according to claim 1, wherein the active ingredient has a solubility in the mixture of solvent, antisolvent and stabilizer of ≤1% by weight based on the total mass.

8. The process according to claim 1, wherein the antisolvent is water.

9. The process according to claim 8, wherein the water has a temperature of >0 to ≤30° C.

10. The process according to claim 1, wherein the solvent is an alkanol.

11. The process according to claim 1, wherein the ratio of the solvent to the antisolvent is ≥1:100.

12. The process according to claim 1, wherein the flow rate of the solvent with the dissolved active ingredient and the flow rate of the antisolvent are in a ratio of ≥1:100 to ≤1:1.

13. The process according to claim 1, wherein the concentration ratio of stabilizer to active ingredient in the mixture is ≤10:1.

14. The process according to claim 1, wherein the stabilizer is polyvinylpyrrolidone.

15. The process according to claim 1, wherein the weight ratio of active ingredient to stabilizer is in a range of ≥1:1 to ≤5:1.
